(19) United States
(12) Patent Application Publication
(10) Pub. No.: US 2003/0163166 A1
(43) Pub. Date: Aug. 28, 2003

(54) Implantable defibrillator design with optimized multipulse waveform delivery and method for using

(76) Inventors: James D. Sweeney, Tempe, AZ (US); Derek J. Dosdall, Mesa, AZ (US); Darrin E. Rothe, Brookfield, WI (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/372,610

(22) Filed: Feb. 21, 2003

Related U.S. Application Data
(60) Provisional application No. 60/361,916, filed on Feb. 27, 2002.

(51) Int. Cl. A61N 1/39

(52) U.S. Cl. 607/5

(57) ABSTRACT

An implantable cardiac ventricular defibrillation system based upon entirely endovascular placement of a minimal number of electrodes is disclosed. The electrodes are designed to deliver a number of subpulses that are rapidly switched within an overall defibrillation shock envelope. The rapid switching between set pairs of electrodes achieves an overall electric field strength and distribution that is optimized for lowest threshold defibrillation energies and voltages. The defibrillation system also incorporates a system and method for optimally tuning and correlating the parameters of the subpulse delivery to the individualized needs of a human subject. The implantable defibrillation system reduces the energy and voltage levels needed for successful ventricular defibrillation in a clinically feasible manner.

### CLAIM TO DOMESTIC PRIORITY

The present non-provisional patent application claims priority to provisional application serial No. 60/361,916, entitled “Implantable Defibrillator Design With Optimized Multipulse Waveform Delivery,” filed on Feb. 27, 2002, by James D. Sweeney.

### FIELD OF THE INVENTION

The present invention relates generally to an implantable defibrillation system, and more specifically, to an implantable cardiac ventricular defibrillation system with entirely endovascular electrode placement and a mechanism for optimal tuning of parameters to individual subjects.

### BACKGROUND OF THE INVENTION

Heart attacks resulting in human death are often due to ventricular fibrillation. Sudden cardiac death accounts for about one-half of all cardiovascular related mortalities in the United States. Approximately 350,000 to 450,000 individuals suffer an out-of-hospital episode of cardiac arrest every year, with less than twenty-five percent surviving a first episode. Approximately one million individuals in the United States develop conditions each year that place them at high risk of sudden death. Ventricular fibrillation is an asynchronous and chaotic activity of the ventricle chambers of the heart. In ventricular fibrillation, the muscle cells of the ventricles begin contracting independently or in an asynchronous manner, rather than in a normal synchronous beat. The result of such asynchronous contracting of the muscle cells is a loss of the pumping function of the heart muscle as a whole, and ultimately circulatory arrest occurs, and the human dies.

One method of reversing ventricular fibrillation and restoring the heart muscle to a normal synchronous beat is through electric shock defibrillation. External defibrillation is the most common method. In external defibrillation, an electric shock is transmitted by applying two plates to the human's chest.

A second method of defibrillation is by using an implantable electric defibrillator that is designed to deliver an electric shock directly to the heart wall. An implantable cardioverter-defibrillator (ICD) can deliver the shock automatically upon detection of ventricular fibrillation. The automatic ICD is an important advance in the treatment of patients at risk of sudden death due to ventricular fibrillation. Approximately 300,000 U.S. patients each year are eligible to receive an ICD device.

From an energy viewpoint, it is advantageous to minimize voltage and current requirements in order to reduce the size of ICDs, as well as increase device lifetime. The amount of energy and voltage required by known implantable defibrillators can cause harm to the patient because the amount of energy currently used can damage structures of the cells. Given that patients receiving ICDs will receive multiple shocks over time, a need exists to develop waveform and electrode strategies that minimize shock strength and energy without decreasing defibrillation effectiveness. It is generally agreed that careful choice of ICD biphasic or triphasic waveform parameters can often yield superior performance in comparison with monophasic waveforms.

Furthermore, the amount of energy and voltage required, along with the number of electrodes and infeasible placement of the electrodes, prevent current implantable defibrillators from being reduced in size to more easily accommodate implantation and be less intrusive in the human body. Finally, current implantable defibrillators have no mechanism for individualizing defibrillation response to a fibrillation event.

Therefore, a need exists for an implantable defibrillator with a minimal number of electrodes, placed in clinically feasible locations with reduced energy and voltage levels to accomplish defibrillation in a system that reduces the size of the ICD while increasing an ICD's safety and efficacy.

### BRIEF DESCRIPTION OF THE DRAWINGS

FIG. 1 is an anterior illustration of a human heart with an implanted defibrillation system according to one embodiment;

FIG. 2 is a posterior illustration of the human heart with the implanted defibrillation system of FIG. 1;

FIG. 3 is a block diagram describing in greater detail an implanted defibrillation system according to one embodiment;

FIGS. 4A-B illustrate various optimized waveforms of one embodiment of the defibrillation system;

FIGS. 5A-B illustrate various optimized waveforms of an alternate embodiment of the defibrillation system; and

FIG. 6 illustrates the defibrillation threshold of the various optimized waveforms of FIGS. 3 and 4.

### SUMMARY OF THE INVENTION

The present invention provides an implantable defibrillation system comprising first and second electrode pathways for delivering a shock, wherein the shock comprises an overall waveform envelope including first and second subpulses, wherein the first and second subpulses are capable of affecting fibrillation of cardiac muscle. The electrode pathways are operatively associated with a system control that is configured for delivering subpulses through the electrode pathways. The overall waveform envelope can be a monophasic, biphasic, triphasic, or other multiphasic waveform. The electrode pathways can be initiated and terminated at several clinically feasible locations.

Cardiac muscle defibrillation can also be individualized according to the present invention. Individualizing cardiac muscle defibrillation includes identifying a parameter influencing cardiac muscle fibrillation and executing a defibrillation response based on the parameter. One parameter that can be used is a strength-duration-time constant and another is the upper level of vulnerability.

Other independent features and advantages of the implantable defibrillation system will become apparent from the following detailed description, taken in conjunction with the accompanying drawings which illustrate, by way of example, the principles of the invention.

### DETAILED DESCRIPTION OF VARIOUS EMBODIMENTS

This description discloses an implantable defibrillation system with an optimized waveform delivery to reduce the amount of energy and voltage needed to achieve defibrillation of the ventricles. The implantable defibrillation system disclosed may be used to treat all forms of cardiac tachyarrythmias, including, but not limited to, ventricular fibrillation and polymorphic ventricular tachycardia.

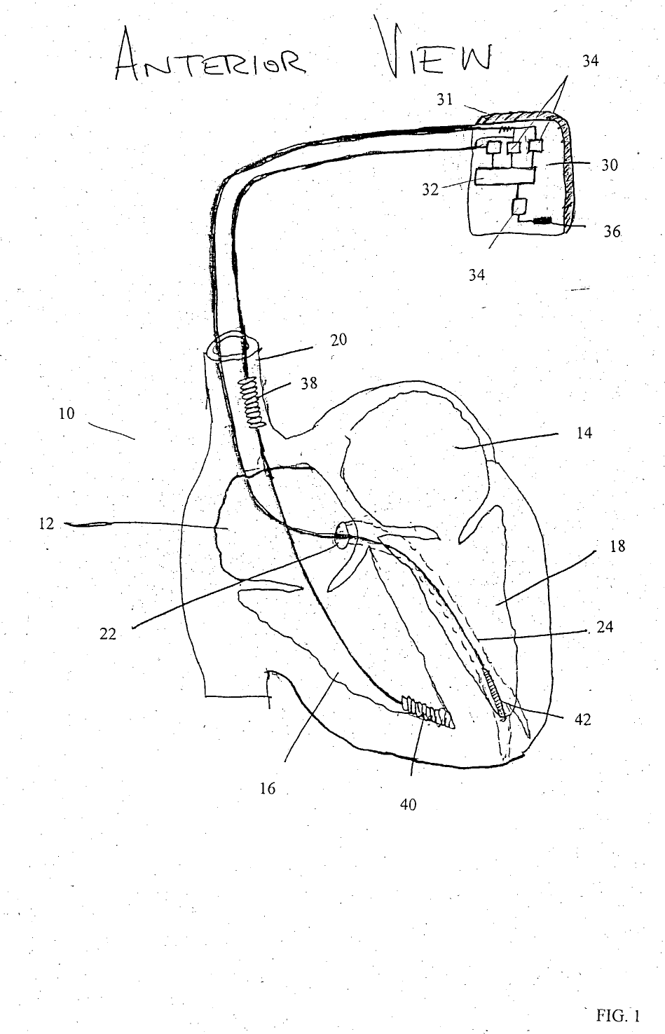

FIG. 1 is an anterior view of one embodiment of the disclosed implantable defibrillation system as implanted in a human heart. The heart 10 is cardiac muscle comprised of four cardiac chambers, the right atrium (RA) 12, the left atrium (LA) 14, the right ventricle (RV) 16, and the left ventricle (LV) 18. FIG. 1 also illustrates other anatomical features of heart 10 including super vena cava (SVC) 20, coronary sinus (CS) 22, and middle cardiac vein (MCV) 24. The heart 10 pumps blood through the body by contraction of the cardiac muscle. The contraction of the cardiac muscle can be detected as an electric signal. Electrical impulses travel in a wave propagation pattern through the atria and then into the ventricles.

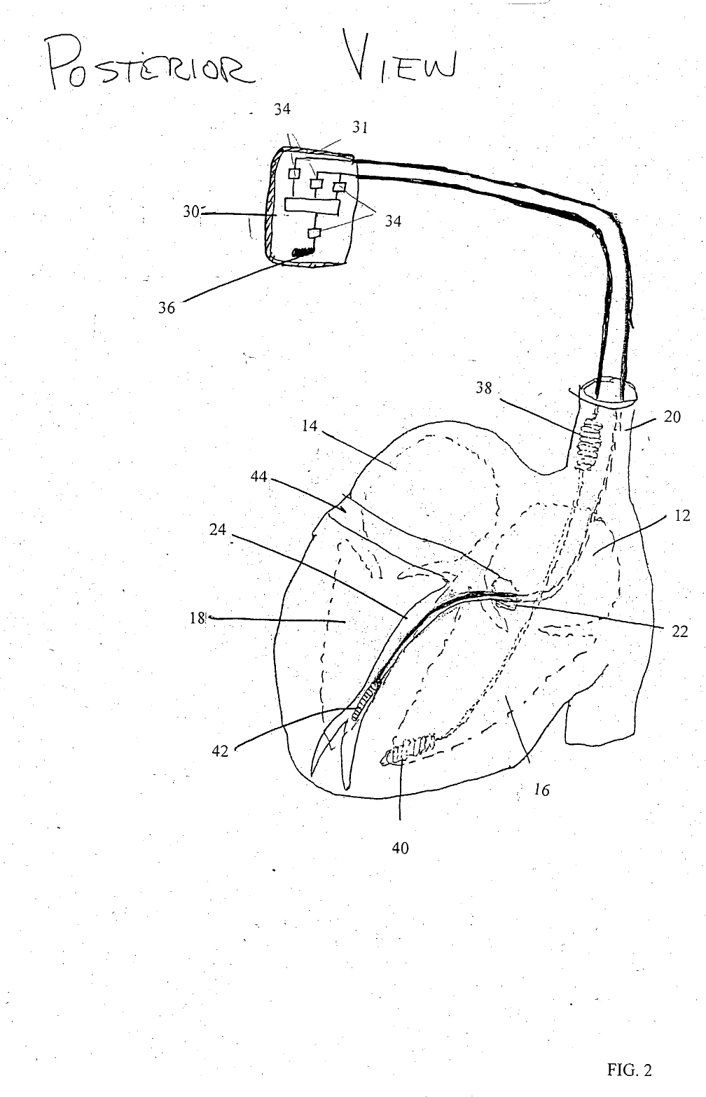

FIG. 2 is a posterior view of the embodiment of the implantable defibrillation system described in FIG. 1. As with FIG. 1, FIG. 2 also schematically illustrates anatomical features of heart 10 including the four chambers, right atrium (RA) 12, left atrium (LA) 14, the right ventricle (RV) 16 and the left ventricle (LV) 18, as well as super vena cava (SVC) 20, coronary sinus (CS) 22, middle cardiac vein (MCV) 24, and the great cardiac vein (GCV) 44.

Referring now to both FIG. 1 and FIG. 2, implantable defibrillator 30 is comprised of an implantable exterior 31 that contains a power source 32 and electronic control circuits 34. Patient electrodes are electronically coupled to electronic control circuits 34. Implantable defibrillator 30 is preferably implanted subcutaneously in the left thoracic region, for example over the left pectoral muscle, of a patient, but can be implanted in other surgically or clinically feasible region.

As illustrated in FIG. 1 and FIG. 2, four patient electrodes are electrically coupled to electronic control circuits 34. According to one embodiment, the patient electrodes are anodes and cathodes capable of forming one or more electrode pathways for delivering a shock comprising an overall waveform envelope. Although the illustrated embodiment as described below uses four patient electrodes, it is recognized that any number of electrodes may be used, creating any number of electrode pathways.

Patient electrodes can be inserted into heart 10 by non-surgical means. A catheter or stylet can be inserted through the superior or inferior vena cava to position the patient electrodes in the proper position in heart 10. The catheter contains patient electrode “leads” or ends. The patient electrode leads can be in the form of coil electrodes, point electrodes, or a combination. Other types of electrodes known in the art may be also be used and are encompassed by the term patient electrodes.

The first electrode, hot can (HC) electrode 36, is the canister of casing of implanantable defibrillator 30, typically implanted over the left pectoral muscle. The second electrode, SVC electrode 38, resides in the superior vena cava 20. The third electrode, RV electrode 40, resides in the right ventricle 16. The fourth electrode, LV electrode 42, is inserted through coronary sinus 22 and resides in middle cardiac vein 24. FIG. 2 illustrates the location of middle cardiac vien 24 on the posterior of heart 10. The patient electrodes are located in the anatomical regions of heart 10 described above due to the clinical feasibility of such locations. Other clinically feasible sites, including, but not limited to, other cardiac veins or arteries, may also be used for electrode location.

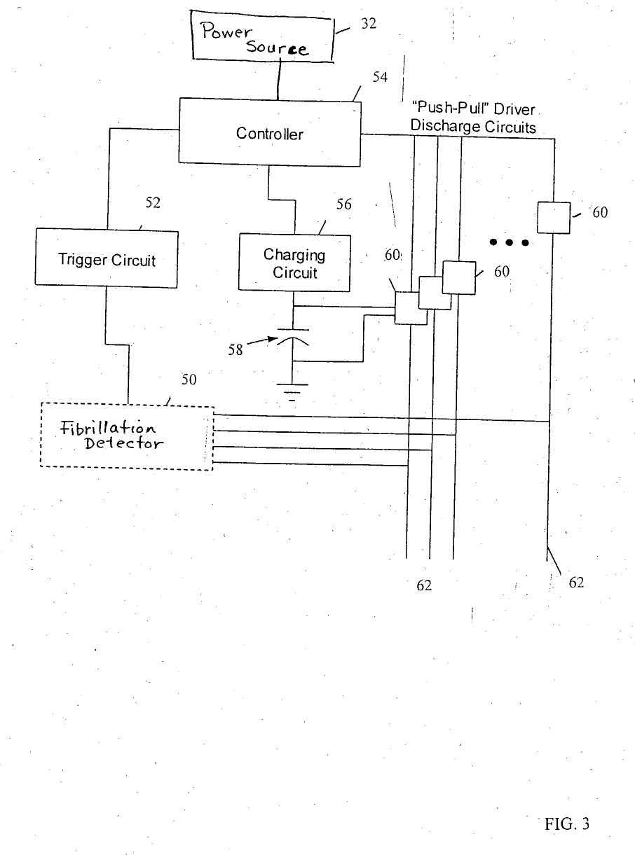

FIG. 3 is a block diagram describing in greater detail the electronic control circuits 34 of the implantable defibrillator system. Depending on the specific application of the defibrillator, fibrillation detector 50 is electronically coupled to patient electrodes 62. Patient electrodes 62 are located in heart 10, as shown in FIG. 1 and FIG. 2. As described in FIG. 1, patient electrodes 62 are not limited to four in number, but any number of electrodes may be used to create one or more electrode pathways for delivering a shock comprising an overall waveform envelope.

Patient electrodes 62 can be coil electrodes, point electrodes, or a combination of coil and point electrodes. As noted in FIG. 1, patient electrodes 62 may also comprise other types of electrodes capable of delivering a defibrillation pulse or sensing fibrillation. Patient electrodes 62 continuously send electrical signals to fibrillation detector 50. Fibrillation detector 50 may be any of several known detectors known to those skilled in the art. Fibrillation detector 50 thus monitors cardiac activity via patient electrodes 62. Thus, fibrillation detector 50 can determine the occurrence of ventricular fibrillation, or other arrhythmia, depending on the application of the implantable device.

Fibrillation detector 50 is electrically coupled to trigger circuit 52. Trigger circuit 52 is electrically coupled to system controller 54. System controller 54 is electrically coupled to power source 32. System controller 54 is also electrically coupled to charging circuit 56. Charging circuit 56 is electrically coupled to capacitor 58. System controller 54 maintains charge on capacitor 58. When commanded by system controller 54, charging circuit 56 charges capacitor 58 from power source 32. Charging circuit 56 also maintains capability for safety discharge of capacitor 58.

Upon detecting fibrillation (or other arrhythmia, depending on the application) fibrillation detector 50 electronically signals trigger circuit 52 to execute a shocking protocol. Trigger circuit 52 accepts the signal to start a shocking sequence and passes the command to system controller 54. System controller 54 then directs charging circuit 54 to charge capacitor 58 from power source 32 to a predetermined voltage. Energy is derived from power source 32 under control of charging circuit 54. Energy is then directed to patient electrodes 62 via discharge circuits 60.

Capacitor 58 holds enough energy to achieve defibrillation. One embodiment uses a 150 microfarad (μF) capacitor over an approximately 50 ohm (Q) load. However, capacitor 58 can range from 10-1000 μF in size and may be a single capacitor or a network of capacitors. The load may also vary, as the actual load is dependant upon the anatomical placement of the patient electrodes 62.

Discharge circuits 60 are electrically coupled and under control of system controller 54. An arbitrary number of discharge circuits 60 may be used in the configuration. Discharge circuits 60 are “push-pull” in nature, in that, at any instant, any given driver can be delivering an anodic or cathodic pathway to patient leads 62. Patient electrodes 62 are the current pathways from discharge circuits 60 to the patient.

Although FIG. 2 illustrates one embodiment of circuitry for an implantable defibrillator, alternate configurations of capacitors and control circuitry may be employed. For example, the power supply may include multiple capacitors. Additionally, the number of patient electrodes 62 may vary from the number shown in FIG. 3. Consequently, the number of discharge circuits 60 will vary accordingly. The position of the electrodes may also be varied to the extent positioning remains clinically feasible. For example, the hot can electrode may be replaced with, supplemented with, or even electrically coupled to one or more of the electrodes residing within the heart, or in other locations in the body.

Fibrillation detector 50 further comprises a system and method for ‘tuning’ the parameters of the high-speed multi-pulse defibrillation system to the needs of an individual subject. The first step in optimizing or tuning the parameters of the defibrillation response is establishing a “strength-duration time constant” for eliciting ectopic beats. Additionally, a measure of the subject's upper limit of vulnerability (ULV) versus the defibrillation threshold (DFT) is key to individualizing the defibrillation response. This measure is based on the established correlation between the defibrillation probability of success curves for rapidly switched shocks and the upper limit of vulnerability probability curves for shocks delivered with the same electrodes and timing. Thus, these key parameters defining the excitability of an individual subject's heart are used to optimize the multi-pulse defibrillation. More specifically, defibrillation can be individualized by adjusting the number of pulses and timing of pulses in the defibrillation response that has the best probability, based on the individual's heart excitability parameters, of successful fibrillation intervention.

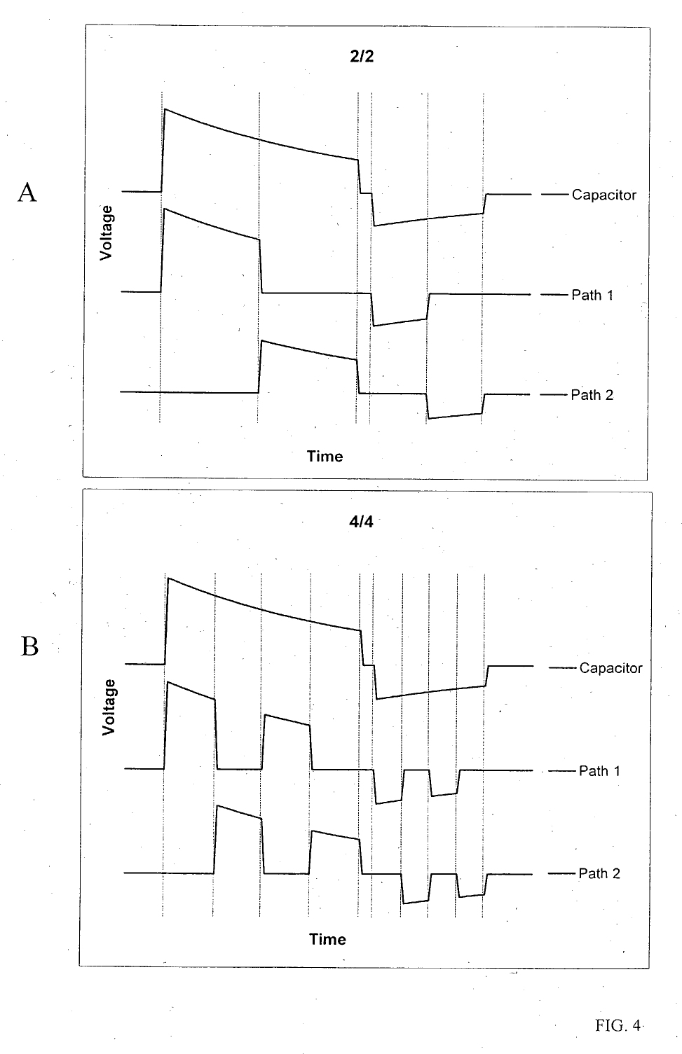

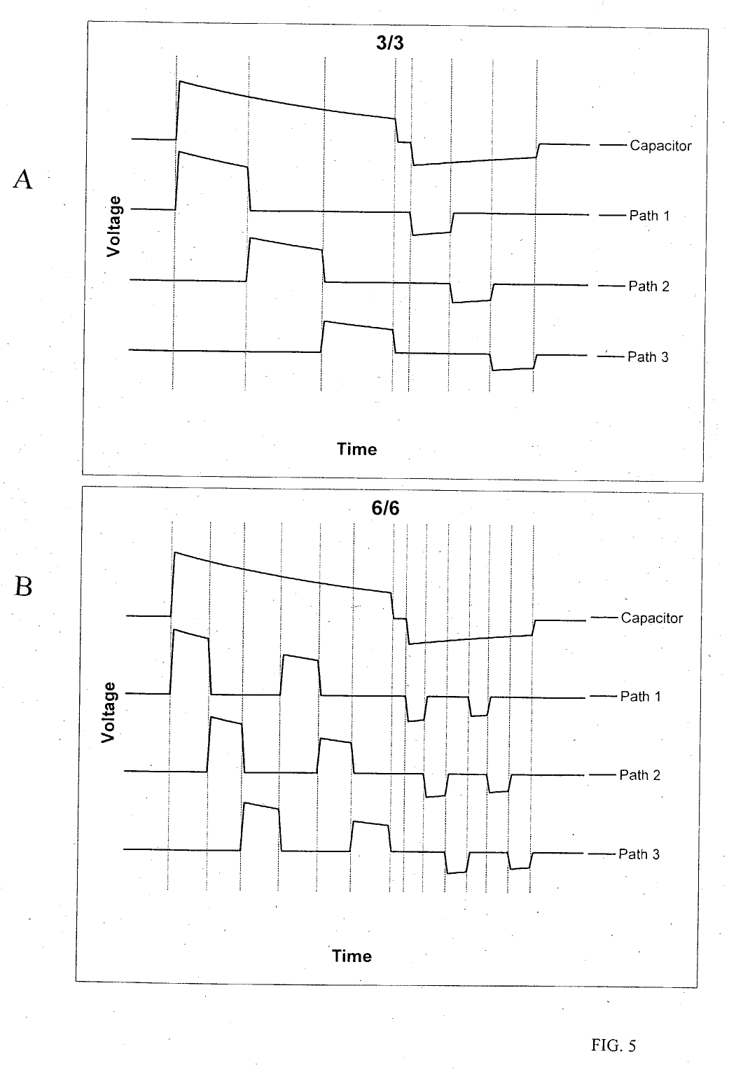

FIGS. 4 and 5 illustrate various multipulse waveforms according to one embodiment. In all waveforms illustrated in FIGS. 4 and 5, a biphasic waveform envelope is employed. However, unlike current biphasic or even triphasic waveforms used in existing implantable defibrillators, each phase of the biphasic or triphasic waveform envelope has two or more subpulses within at least one phase of the waveform envelope. Additionally, the subpulses may be generated through more than one electrode pathway.

In FIGS. 4 and 5 the subpulses are generated using interleaved pulses, also known as sequential pulses, as created by one or more electrode pathways. In other words, each pathway generates one or more subpulses in sequence. Further, as illustrated below, the sequence may be repeated one or more times to generate a greater number of subpulses within each pulse.

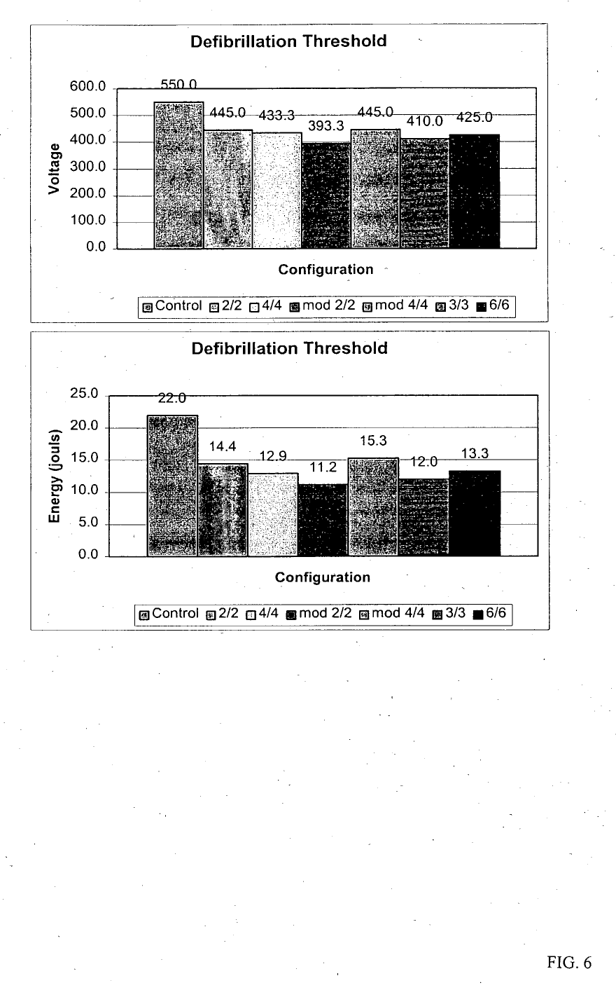

In FIGS. 4 and 5, the biphasic waveform of the capacitor in each graph illustrates a control defibrillation absent an optimized multipulse waveform defibrillation. The time for each waveform graph is measured in milliseconds (ms). The first phase for each biphasic waveform is 7 ms and the second phase in the biphasic waveform is 4 ms with an interpulse of 0.5 ms. However, it is recognized that the time for each phase can range from 1-10 ms, with an interpulse period or separation of 100 μs to 10 ms. The voltage used in FIGS. 4 and 5 is several hundred volts. However, the voltage can range between 100 and 1000 volts, depending on the optimization of the multipulse, as described in FIG. 6. However, for other arrhythmias, a lower voltage, even below 100 volts, can be used.

As shown in FIGS. 4 and 5, each electrode pathway is pulsed for approximately an equal amount of time, equating to an approximate equal division of the phase between subpulses. However, it is also recognized that an individual subpulse time can be any fraction of time of the entire pulse time, and that the time for each subpulse need not be equally distributed among the number of subpulses.

It is further recognized that while the subpulse pattern illustrated in FIGS. 4 and 5 is applied to a biphasic waveform envelope, the advantages of subpulsing in clinically feasible cardiac regions can be applied to a monophasic, triphasic, or other multiphasic (four or more phases) overall waveform envelope. The triphasic or other multiphasic waveform envelopes may or may not utilize subpulses in every phase, depending on the fibrillation response protocol, duration of the phase, or other parameters. However, one embodiment of optimized multipulse waveforms envisions employing subpulses in at least one phase of a the overall waveform envelope.

In both FIGS. 4A and 4B, two electrode pathways are used to generate subpulses in multiples of two. In Path 1, RV electrode 40 is the cathode and hot can electrode 36 is the anode. In an alternate embodiment, hot can electrode 36 is electrically coupled with SVC electrode 38 and used as the anode. In Path 2, LV electrode 42 is the cathode and SVC electrode 38 is the anode. Again, in an alternate embodiment, SVC electrode 38 is electrically coupled with hot can electrode 36 and used as the anode.

As noted previously, the electrode pathways are not limited in number, nor in electrode pathway configuration, to those electrode pathways illustrated in FIG. 4. Therefore, in using two electrode pathways as illustrated in FIG. 4, it is recognized that subpulses may be generated in any number that is a multiple of two merely by repeating the electrode pathway sequence the desired multiple of times within each phase of the overall biphasic waveform envelope.

FIG. 4A illustrates a multipulse waveform according to one embodiment which employs two electrode pathways, each generating one subpulse in each phase of the overall biphasic waveform envelope. Thus, each phase of the waveform envelope has two subpulse. FIG. 4B illustrates an alternate embodiment of a multipulse waveform also employing two electrode pathways, but with each electrode pathway generating two interleaved subpulses in each phase of the overall biphasic waveform envelope resulting in four subpulses in each phase.

In both FIGS. 5A and 5B, three electrode pathways are used to generate subpulses in multiples of three. In Path 1, RV electrode 40 is the cathode and hot can electrode 36 is electrically coupled with SVC electrode 38 and used as the anode. In an alternate embodiment, either hot can electrode 36 or SVC electrode 38 alone is used as the anode. In Path 2, hot can electrode 36 is the cathode and LV electrode 42 is the anode. In Path 3, LV electrode 42 is the cathode and RV electrode 40 is the anode. As in FIG. 4, the electrode pathways are not limited in number, nor in electrode pathway configuration, to the illustrated embodiments. Rather, various combinations of electrode pathways can be used.

FIG. 5A illustrates a multipulse waveform according to one embodiment which employs three electrode pathways, each generating one subpulse in each phase of an overall biphasic waveform envelope. Thus, each phase of the waveform envelope has three subpulses. FIG. 5B illustrates an alternate embodiment of a multipulse waveform also employing three electrode pathways with each electrode pathway generating two interleaved subpulses. Thus the system generates six subpulses in each phase of the overall biphasic waveform envelope.

FIG. 6 illustrates experimental results achieved with one embodiment of an implantable defibrillator according to this description. In this non-limiting example, pigs were initially anesthetized using 4 to 6 mg/lb of Telezol IM (with 2.2 mg/kg xylazine), intubated, and then maintained on a large animal anesthesia-ventilator using gaseous isoflurane (approximately 1.5 to 2%) with oxygen using aseptic (sterile) surgical procedures. Succinylcholine (1.5 mg/kg initial intravenous dose followed by 0.5 mg/kg intravenous infusions every 20 min) was used to produce adequate muscle relaxation. One carotid artery will be cannulated to allow monitoring of arterial blood pressure. Lead II EKG was also monitored. Rectal temperature will be measured and maintained within normal values. Ringers lactate supplemented with sodium bicarbonate will be infused continuously through a venous line. Blood gases, partial pressure of oxygen and carbon dioxide, will be analyzed at least every 30 minutes.

An electrode (4 cm length, 1 mm diameter, wound 80/20 Pt—Ir wire) was inserted into the posterior cardiac vein (i.e. electrode ‘LV’). Another defibrillation catheter was inserted via the right jugular with the distal shocking coil (5 cm length, 1 cm circumference) advanced into the right ventricular apex (i.e. electrode ‘RV’), and with a second coil (7 cm length, 1 cm circumference) on the same catheter placed in the superior vena cava (i.e. electrode ‘SVC’). The RV coil and the SVC coil had a distance of 9 cm between them on the catheter. A mock sub-cutaneous ‘can’ electrode (simulating the active can electrode of an actual ICD implant) was placed on the left lateral thorax (i.e. electrode ‘can’).

Fibrillation was induced with a 60 Hz square wave delivered for 2 seconds through the RV pacing tip. Following 10 seconds of fibrillation, a test shock was delivered. If the test shock was unsuccessful at defibrillating the animal, a higher voltage shock was immediately delivered to rescue the animal. The 50% defibrillation threshold (DFT 50, or the shock strength that defibrillates the heart approximately 50% of the time) was approximated using a standard up/down bracketing protocol.

In FIG. 6, the control waveform did not utilize an optimized multipulse waveform envelope. The control waveform is illustrated in FIGS. 4 and 5 as the capacitor waveform. The control waveform required the greatest amount of energy, 22 joules, to achieve defibrillation. However, less energy was required to accomplish defibrillation using optimized multipulse waveform envelopes described above. In FIG. 6, the waveforms used correspond to those described in FIGS. 4 and 5.

As shown in FIG. 6, defibrillation using the optimized multipulse waveform according to the embodiments described above required thirty to fifty percent less energy to accomplish defibrillation than the control waveform. Therefore, defibrillation according to the embodiments described above can successfully accomplish defibrillation with lower voltage levels. Thus, the disclosed implantable defibrillator can reduce potential harm to patients by higher voltage levels. Further, requiring less energy can increase the lifetime of a defibrillator device, resulting in less replacement and invasive procedures on a patient. Finally, the size of defibrillator devices can also be minimized due to lower voltage requirements.

The optimized multipulse waveform described above may also be used in external defibrillation. Patient electrodes can be attached to various dermal regions, for example, on the thoracic region and the torso region, including below the axilla and above the nipple. Defibrillation utilizing subpulses in one or more phases of a biphasic or triphasic waveform is accomplished in a similar manner as described above except with the patient electrodes and defibrillator located externally.

Various embodiments of the invention are described above in the Drawings and Description of Various Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

What is claimed is:

1. A cardiac defibrillation system comprising:
a first electrode pathway configured for delivering a shock along a first predetermined current path, wherein the shock comprises an overall waveform envelope including a first subpulse, wherein the first subpulse is capable of affecting fibrillation of cardiac muscle;
a system control operatively associated with the first electrode pathway, wherein the system control is configured for delivering subpulses along electrode pathways; and
a second electrode pathway operatively associated with the system control, configured for delivering a shock along a second predetermined current path, wherein the shock comprises an overall waveform envelope including a second subpulse, wherein the second subpulse is capable of affecting fibrillation of cardiac muscle and wherein the second subpulse has a polarity the same as the first subpulse.

2. The cardiac defibrillation system of claim 1, wherein the overall waveform envelope is a monophasic waveform envelope.

3. The cardiac defibrillation system of claim 1, wherein the overall waveform envelope is a biphasic waveform envelope.

4. The cardiac defibrillation system of claim 1, wherein the overall waveform envelope is a triphasic waveform envelope.

5. The cardiac defibrillation system of claim 1, wherein the first electrode pathway includes an electrode positioned in the thoracic region of a mammal.

6. The cardiac defibrillation system of claim 1, wherein the first electrode pathway includes an electrode positioned in the superior vena cava of a mammal.

7. The cardiac defibrillation system of claim 1, wherein the first electrode pathway includes an electrode positioned in the right ventricle of a mammal.

8. The cardiac defibrillation system of claim 1, wherein the first electrode pathway includes an electrode positioned in the middle cardiac vein of a mammal.

9. The cardiac defibrillation system of claim 1, wherein the first electrode pathway includes an electrode positioned on the dermis of a mammal.

10. The cardiac defibrillation system of claim 1, wherein the first electrode pathway is configured for delivering a shock along a first predetermined current path, wherein the shock comprises an overall waveform envelope including a third subpulse, wherein the third subpulse has a polarity opposite the first subpulse.

11. The cardiac defibrillation system of claim 10, wherein the second electrode pathway is configured for delivering a shock along a second predetermined current path, wherein the shock comprises an overall waveform envelope including a fourth subpulse, wherein the fourth subpulse has a polarity opposite the second subpulse.

12. A method for intervening in cardiac muscle fibrillation comprising:
positioning a plurality of electrodes in a mammal;
configuring a first electrode pathway for delivering a shock along a first predetermined current path, wherein the shock comprises an overall waveform envelope including a first subpulse, wherein the first subpulse is capable of affecting fibrillation of cardiac muscle; and
configuring a second electrode pathway for delivering a shock along a second predetermined current path, wherein the shock comprises an overall waveform envelope including a second subpulse, wherein the second subpulse is capable of affecting fibrillation of cardiac muscle and wherein the second subpulse has a polarity the same as the first subpulse.

13. The method of claim 12, wherein the overall waveform envelope is a monophasic waveform envelope.

14. The method of claim 12, wherein the overall waveform envelope is a biphasic waveform envelope.

15. The method of claim 12, wherein the overall waveform envelope is a triphasic waveform envelope.

16. The method of claim 12, wherein the first electrode pathway includes an electrode positioned in the thoracic region of a mammal.

17. The method of claim 12, wherein the first electrode pathway includes an electrode positioned in the superior vena cava of a mammal.

18. The method of claim 12, wherein the first electrode pathway includes an electrode positioned in the right ventricle of a mammal.

19. The method of claim 12, wherein the first electrode pathway includes an electrode positioned in the middle cardiac vein of a mammal.

20. The method of claim 12, wherein the first electrode pathway includes an electrode positioned on the dermis of a mammal.

21. A method for individualizing cardiac muscle defibrillation comprising:
identifying a parameter influencing cardiac muscle fibrillation; and
executing a defibrillation response based on the parameter.

22. The method of claim 21, wherein the parameter influencing cardiac muscle fibrillation is a strength-duration-time constant.

23. The method of claim 21, wherein the parameter influencing cardiac muscle fibrillation is an upper level of vulnerability.

24. A cardiac defibrillation system comprising:
a first electrode pathway configured for delivering a shock along a first predetermined current path, wherein the shock comprises an overall waveform envelope including a first subpulse and a second subpulse, wherein the first subpulse is capable of affecting fibrillation of cardiac muscle and wherein the first subpulse has a polarity the same as the second subpulse; and
a system control operatively associated with the first electrode pathway, wherein the system control is configured for delivering subpulses through the first electrode pathway.

25. The cardiac defibrillation system of claim 24, wherein the first electrode pathway is configured for delivering a shock along a first predetermined current path, wherein the shock comprises an overall waveform envelope including a third subpulse, wherein the third subpulse has a polarity opposite the first subpulse.
